(19) United States
(12) Patent Application Publication
(10) Pub. No.: US 2016/0015531 A1
(43) Pub. Date: Jan. 21, 2016

(54) Structure of Tumble-Resistant Knee Joint

(71) Applicants: Chia-Pao Cheng, New Taipei City (TW); Hsiang-Ming Wu, New Taipei City (TW)

(72) Inventors: Chia-Pao Cheng, New Taipei City (TW); Hsiang-Ming Wu, New Taipei City (TW)

(21) Appl. No.: 14/476,741

(22) Filed: Sep. 4, 2014

(30) Foreign Application Priority Data
Jul. 18, 2014 (CN) 201410350319.7

(51) Int. Cl. A61F 2/64

(52) U.S. Cl. CPC A61F 2/64

(57) ABSTRACT

A tumble-resistant knee joint includes a knee carrier, a main base, and a coupler connected to each other. The knee carrier includes a connection piece and two connection bars to couple to an upper portion of the main base. The coupler includes coupler recesses formed in one side thereof corresponding to extensions of grips to receive push blocks therein and has two coupling bolts extending through a lower portion of the main base to allow the coupler connected to the lower portion of the main base. The main base receives therein an axle, which is provided, respectively and in sequence from a center thereof toward two opposite ends, with two bearings, two one-way bearings, two bushings, and two grips. The grips include extensions that cooperate with the push blocks to selectively grip or release the axle. The one-way bearings constrain the rotation of the axle to be in one direction.

### (a) TECHNICAL FIELD OF THE INVENTION

The present invention generally relates to an improved structure of a tumble-resistant knee joint, and more particularly to a structure that is applicable to a thigh socket of a limb prosthesis and, when used in a walking motion of a knee joint of a user, provides a function of preventing tumble through dual constraints achieved with one-way bearings used in combination of an axle and grips and push blocks abutting each other.

### (b) DESCRIPTION OF THE PRIOR ART

For users of thigh support sockets of limb prostheses, the limb prostheses may be classified as: above-knee prostheses, which do not contain bones, ligaments, and muscles that a healthy knee may contain for driving a complicated connection between a thigh and a shank and only relies upon the residuum of a leg to apply a force in a small range. A seemingly easy motion, for example stretching exercise, such as bending and stretching the knee joint, partly squatting, and sitting down, is difficult or even dangerous for users of this type. This is because the knee joint and all the parts below the knee joint are metallic parts and metallic parts cannot be made in the form of inter-related organizations, such as ligaments, muscles, and tendons. Thus, to prevent the shank side of a limb prosthesis from bending down unexpectedly to lead to a knee-down situation during walking is one of issues to be first improved by the present invention.

In the known structures, the most commonly used solution is to constrain or stop further rotation of an axle through an angle induced by various motions of the prosthesis user. An example is Taiwan Utility Model M284363, which achieves the purpose of resisting tumble by applying braking induced by a one-way bearing. Another example is Taiwan Patent No. I388314, which makes use of grips to achieve releasing and clamping in order to constrain the rotation of an axle within a predetermined range thereby preventing the occurrence of an accident caused by unexpected bending of a prosthetic knee joint.

From the above-described conventional structures, it can be known that a knee joint of limb prosthesis uses the body weight of a user or a reaction force induced by contact with the ground to constrain an axle so as to achieve the desired purpose and grips are considered the primary constraining structure. Wearing of the grips, after long use and increased number of operations, may often cause situations of abrasion, flexing deformation, and breaking. Thus, different constraining structures or components may be additionally used to spread the pressure applied thereto in order achieve improvements of the above-discussed problems.

### SUMMARY OF THE INVENTION

The present invention provides an improved structure of tumble-resistant knee joint, which is made up of a knee carrier, a main base, and a coupler connected to each other. The knee carrier generally comprises a connection piece and two connection bars to achieve coupling with an upper portion of the main base. The coupler comprises two coupler recesses formed in one side thereof to receive push blocks disposed therein and two coupling bolts extending through a lower portion of the main base to allow the coupler to couple to the underside of the main base. The main base comprises an axle arranged therein and the axle is provided, in sequence from a center thereof toward two opposite ends, with two bearings, two one-way bearings, two bushings, and two grips. The two grips each comprise an extension cooperating with the above discussed push block and the two one-way bearings constrain the rotation of the axle to be in one direction so as to constrain, through the connection piece and the connection bars, the rotation angle of the axle.

Compared to the drawbacks of the prior art, the present invention uses one-way bearings to constrain the rotation direction of an axle and takes advantage of gripping and releasing of grips to allow the present invention to impose limitation to a rotation structure of the axle in such a way that the one-way bearings and the grips collaborate with each other to achieve such constraint and thus overcome the problems of the prior art and also help enhance safety of a walking motion to allow a user of a limb prosthesis to stand and bend to walk in a more concern-free manner.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of the present invention.

FIG. 2 is an exploded view of the present invention.

FIG. 3 is a cross-sectional view of the present invention.

FIG. 4 is a schematic view showing the present invention in an upright condition.

FIG. 4A is an enlarged view of a portion of a side of the present invention in the upright condition.

FIG. 5 is a schematic view showing the present invention in a bending condition of standing.

FIG. 5A is an enlarged view of a portion of a side of the present invention in the bending condition of standing.

FIG. 6 is a schematic view showing the present invention in a start-to-bend condition.

FIG. 6A is an enlarged view of a portion of a side of the present invention in the start-to-bend condition.

FIG. 7 is a schematic view showing the present invention in an upright condition in a walking motion.

FIG. 7A is an enlarged view of a portion of a side of the present invention in the upright condition in a walking motion.

FIG. 8 is a schematic view showing the present invention in a bending condition in a walking motion.

FIG. 8A is an enlarged view of a portion of a side of the present invention in the bending condition in a walking motion.

FIG. 9 is a schematic view showing the present invention in a start-to-bend condition in a walking motion.

FIG. 9A is an enlarged view of a portion of a side of the present invention in the start-to-bend condition in a walking motion.

FIG. 10 is a schematic view showing the present invention in a return-to-upright condition in a walking motion.

FIG. 10A is an enlarged view of a portion of a side of the present invention in the return-to-upright condition in a walking motion.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

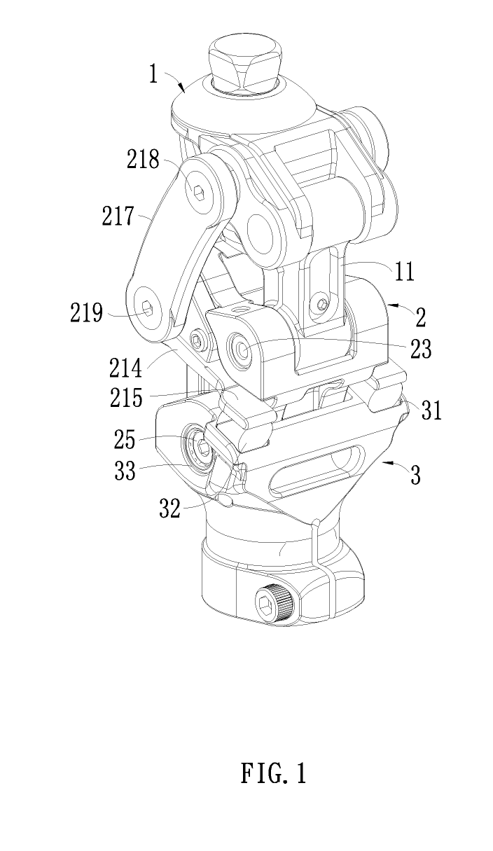

Referring to FIGS. 1-6A, the present invention provides an improved structure of tumble-resistant knee joint, which is arranged between a thigh socket (4) and shank frame (41) of a limb prosthesis and comprises a knee carrier (1) that comprises a rotatable connection piece (11) and a through hole (13) and a connection bar (217) arranged beside each of two ends of the through hole (13), the connection piece (11) comprising a shaft (12); a main base (2), which comprises a shaft coupling hole (23) that is provided for coupling with the shaft (12) and an axle hole (21) formed in an upper portion thereof, the main base (2) having a lower portion in which a first threaded hole (24), the axle hole (21) receiving an axle (211) to extend therethrough with two bearings (210), two one-way bearings (212), two bushings (213), and two grips (214) respectively and sequentially fit over opposite sections of the axle (211) extending from a center towards two opposite ends, wherein the one-way bearings (212) constrain the axle (211) to rotate in one predetermined direction and the grips (214) each comprise an extension (215), the two connection bars (217) of the knee carrier (1) being respectively set outside of the two grips (214) in a coupled condition; and a coupler (3), which comprises two coupler recesses (31) formed in a top thereof, the coupler (3) having two sides in which two second threaded holes (33) are respectively formed, the coupler (3) receiving therein an elastic element (34) that provides cushioning to the shank frame (41) arranged therebelow and connected thereto, two coupling bolts (25) being respectively received through the second threaded holes (33) to mate and couple to the first threaded hole (24), the two coupler recesses (31) each receiving and retaining therein a push block (32), whereby when the coupler (3) is in operation in walking, the connection bars (217) drive the axle (211) that is coupled thereto to cause the main base (2) to rotate and applies forces via the extensions (215) of the grips (214) to the push blocks (32) the coupler (3) in order to tightly grip the one-way bearings (212) thereby constraining the rotation direction of the axle (211) so that the rotation angle of the axle (211) driven by the connection bars (217) is constrained, meaning bending of the knee joint is constrained.

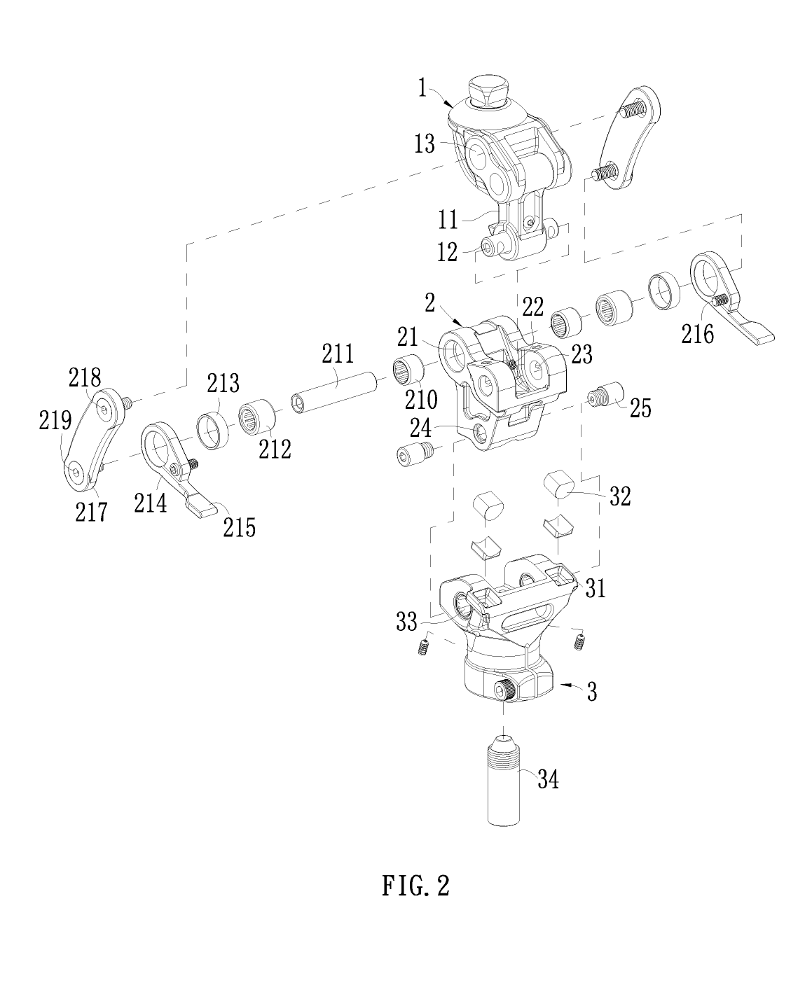

Referring to FIGS. 1-2, the knee carrier (1), the main base (2), and the coupler (3) are each composed of a left side portion and a right side portion of which the structures are identical so that in the following, only one of the two side portions will be described for simplicity. The main base (2) further comprises a threaded hole (22) between the axle hole (21) and the shaft coupling hole (23) thereof. The threaded hole (22) provides a function of screwing engagement with a bolt (216) provided on the grip (214) for purposes of fixing. The connection bar (217) has an upper end in which an upper through hole (218) is formed for coupling with the through hole (13) of the knee carrier (1) and the connection bars (217) has a lower end in which a lower through hole (219) is formed for coupling with the main base (2). The coupler (3) receives there in the elastic element (34) and the elastic element (34) provides a function of assisting the knee joint to stretch.

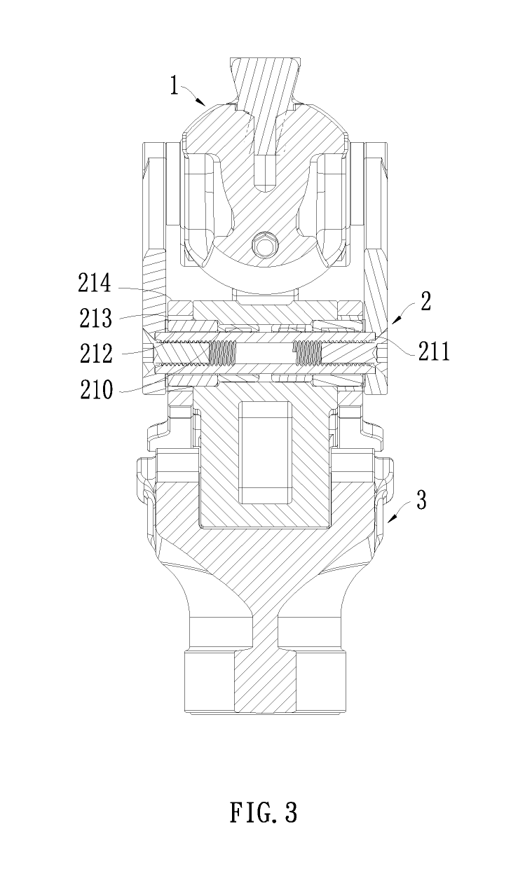

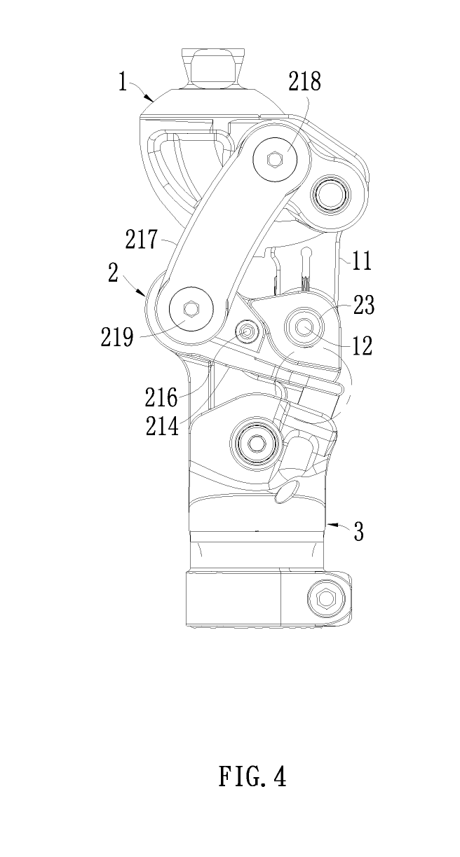

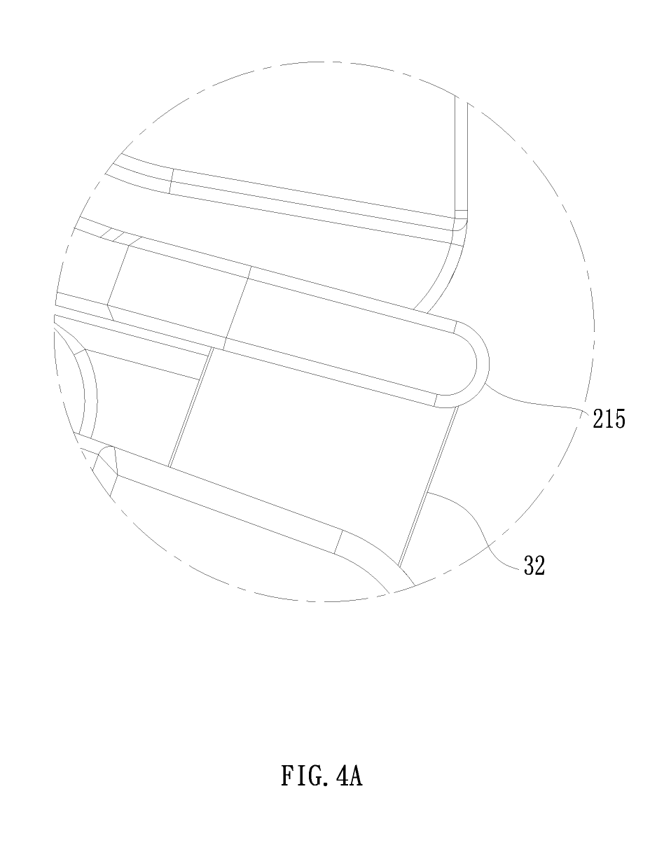

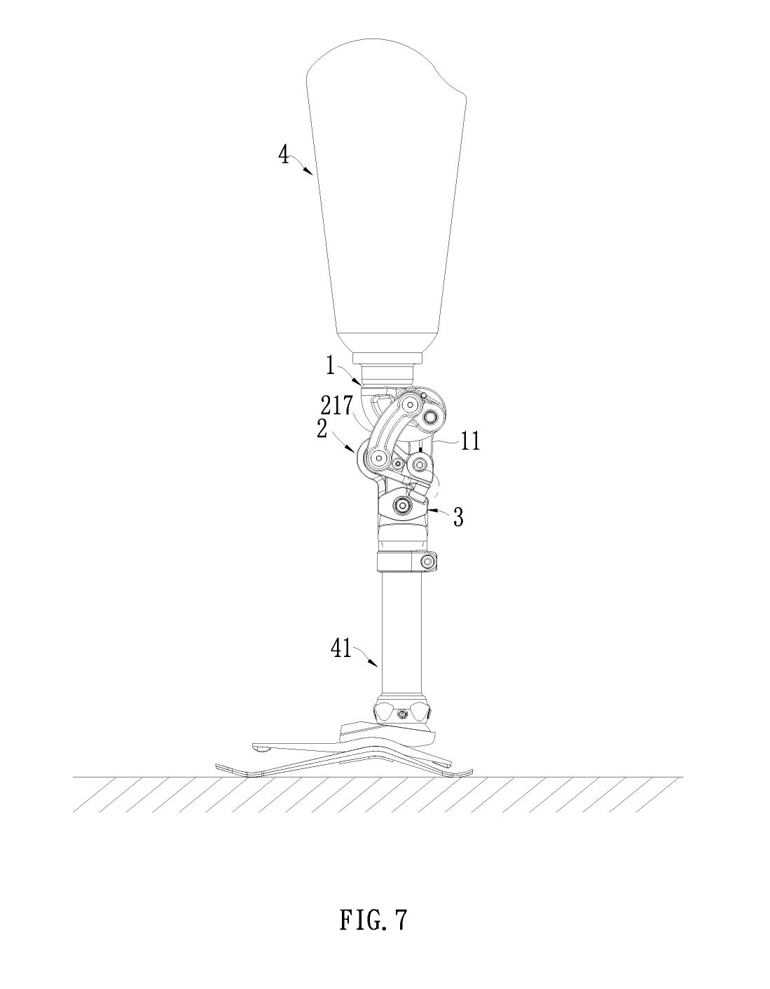

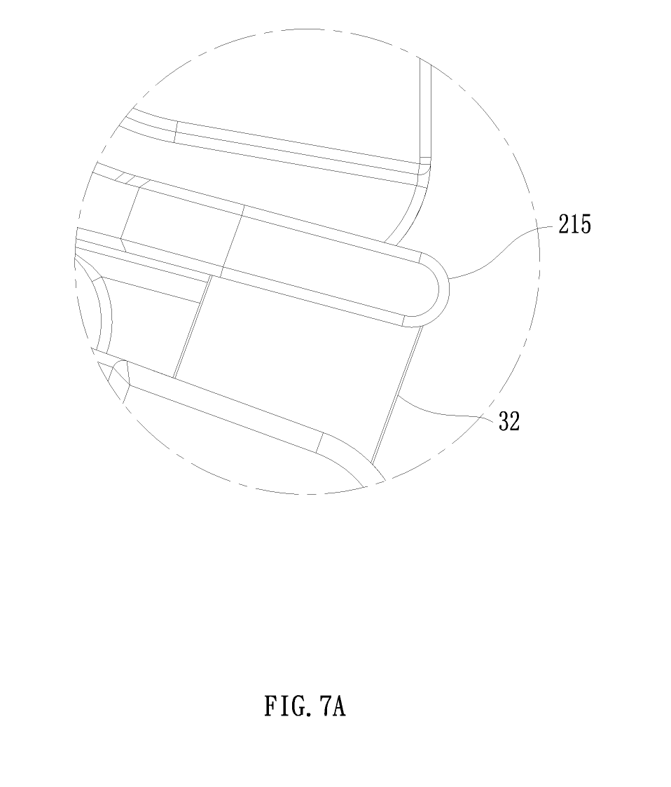

Referring to FIGS. 3-10A, the grips (214) that are arranged at the two external portions of the axle (211), as shown in FIG. 3, are each provided, in sequence from the outside, with the bushing (213), the one-way bearing (212), and the bearing (210) in order to have the rotation angle of the axle (211) constrained thereby. Further as shown in FIGS. 4, 4A, 7, and 7A, in an upright condition in a walking motion, the prosthesis thigh socket (4) located above the knee carrier (1) and the shank frame (41) located below the coupler (3) are both perpendicular to a horizontal surface of the ground and at this moment, the axle (211) and the grips (214) that are arranged in the interior are in an initial state.

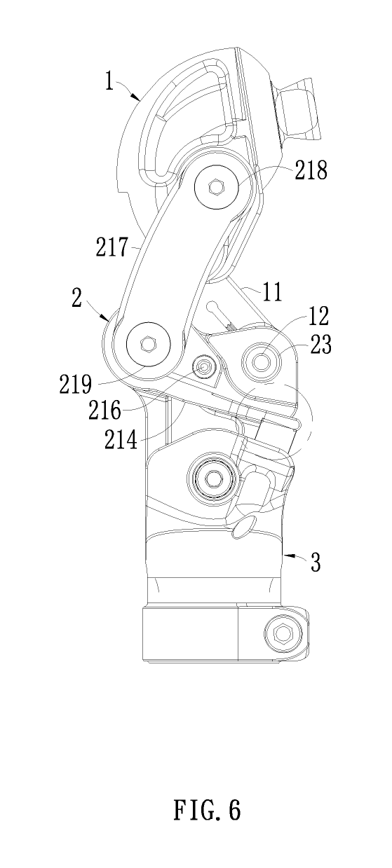

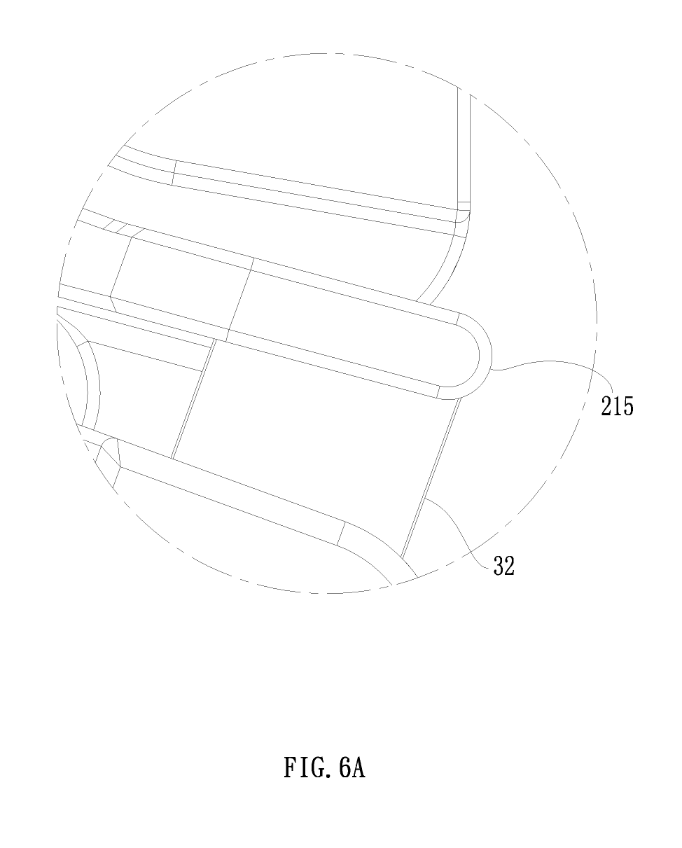

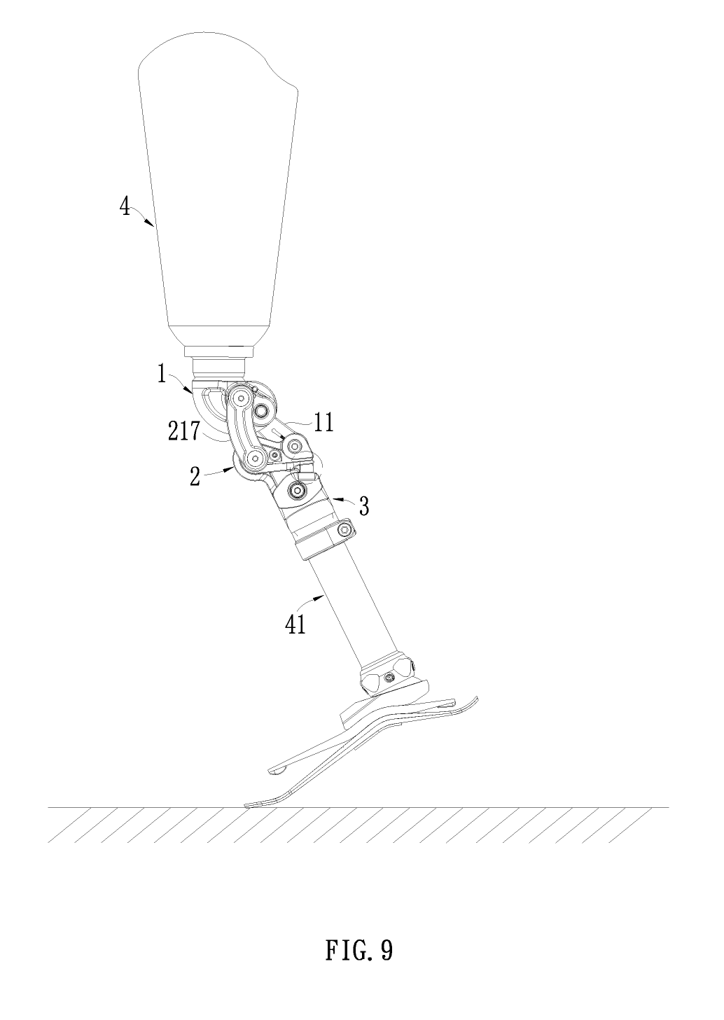

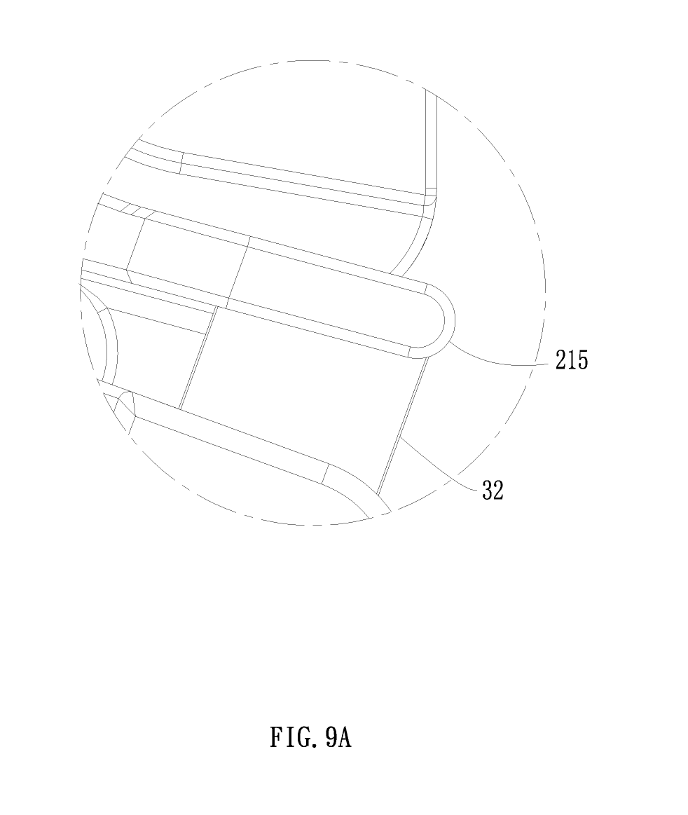

In a condition of starting to bend in the walking motion, as shown in FIGS. 6, 6A, 9, and 9A, the center of gravity of the limb prosthesis is shifted, making the force that is applied by the main base (2) to the push block (32) varnish thereby the grips (214) no long tightly grips the one-way bearings (212), allowing the knee joint to bend for accomplishing the purpose of walking.

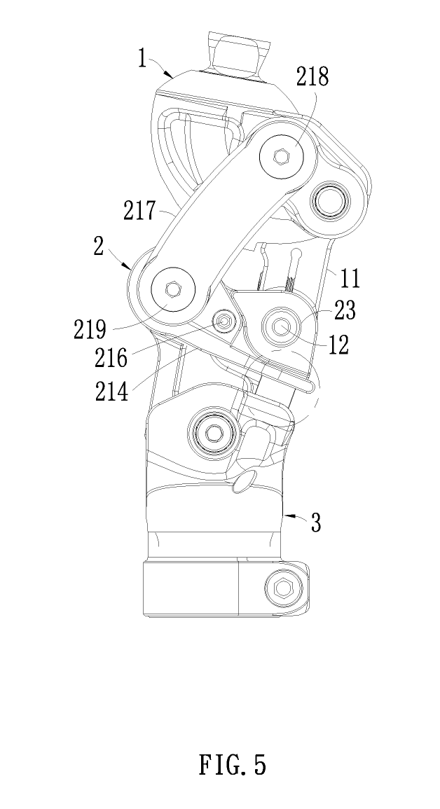

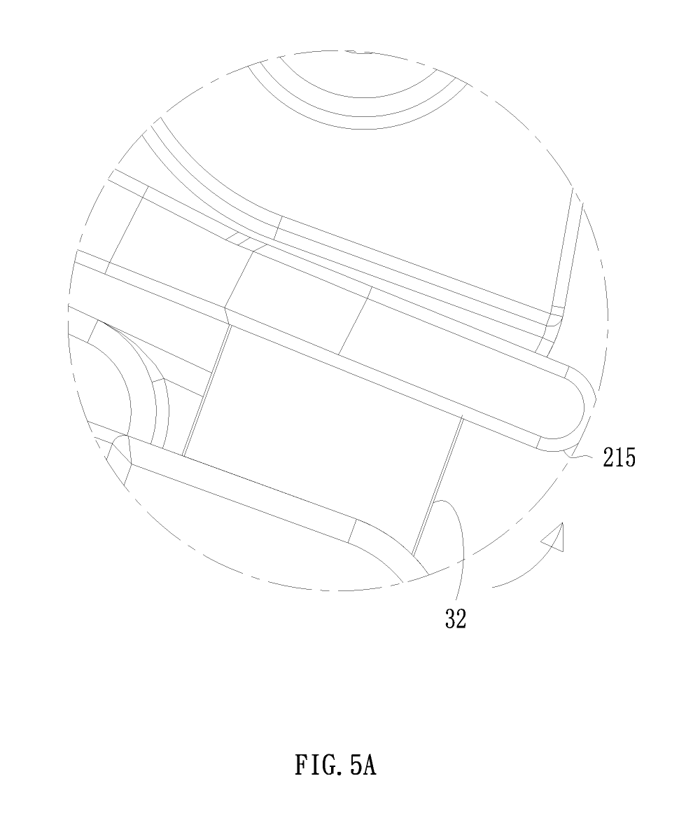

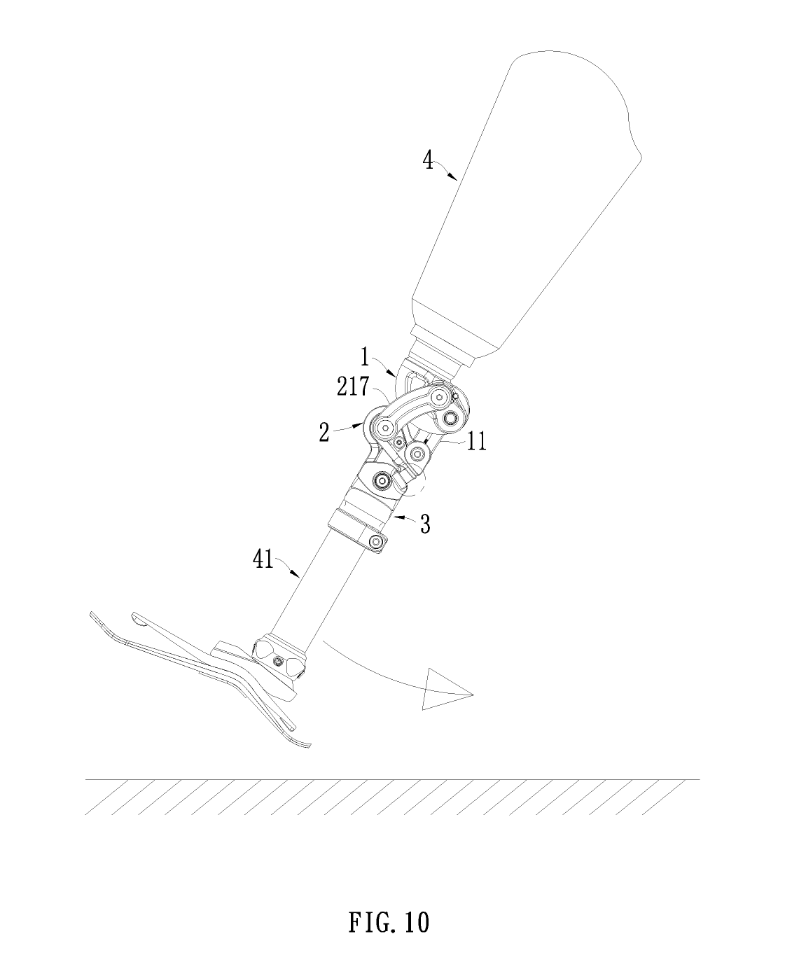

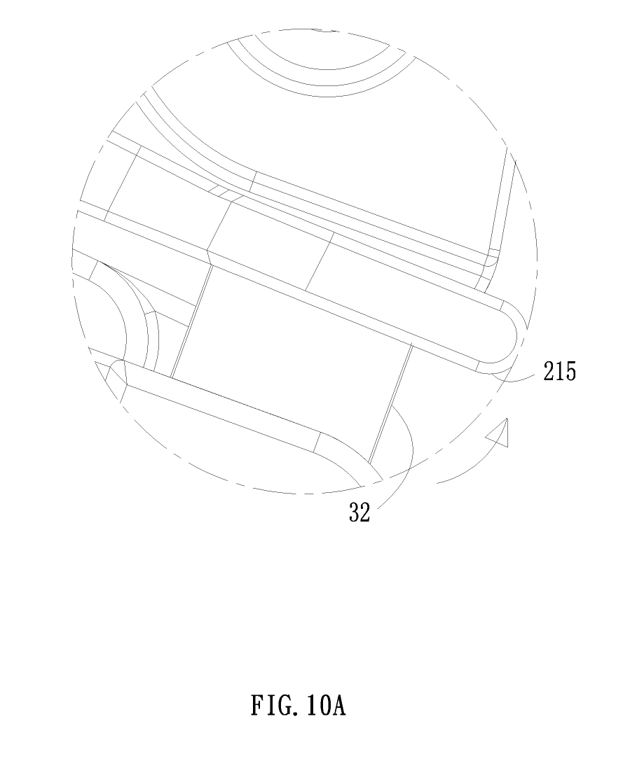

In returning back to the upright condition in the walking motion, the limb prosthesis shows a stretched condition, and as shown in FIGS. 5, 5A, 10, and 10A, due to the effect of gravity, the coupler (3), the shank frame (41), and a sole that are located on the lower side would use the second threaded holes (33) as a rotation axis to make the push blocks (32) push against the extensions (215) of the grips (214) so as to set the one-way bearings (212) in a tightly gripped state and also impose constrain to the rotation direction of the axle (211) to thereby prevent the prosthesis shank from bending downward unexpectedly to cause a situation of kneeing down during walking.

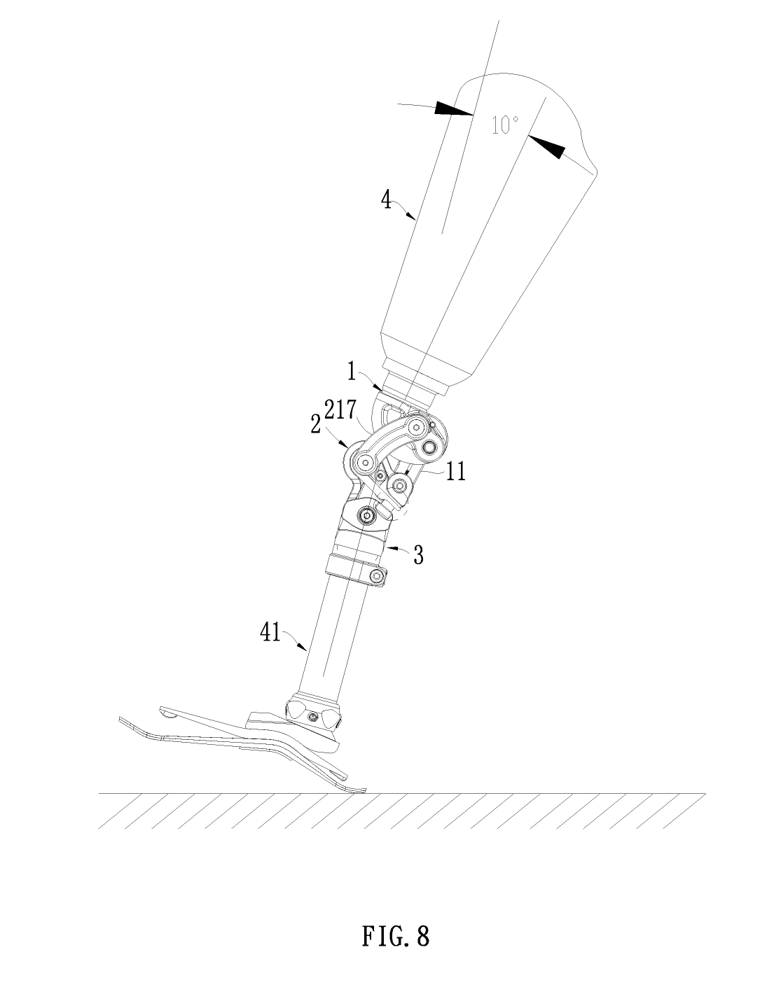

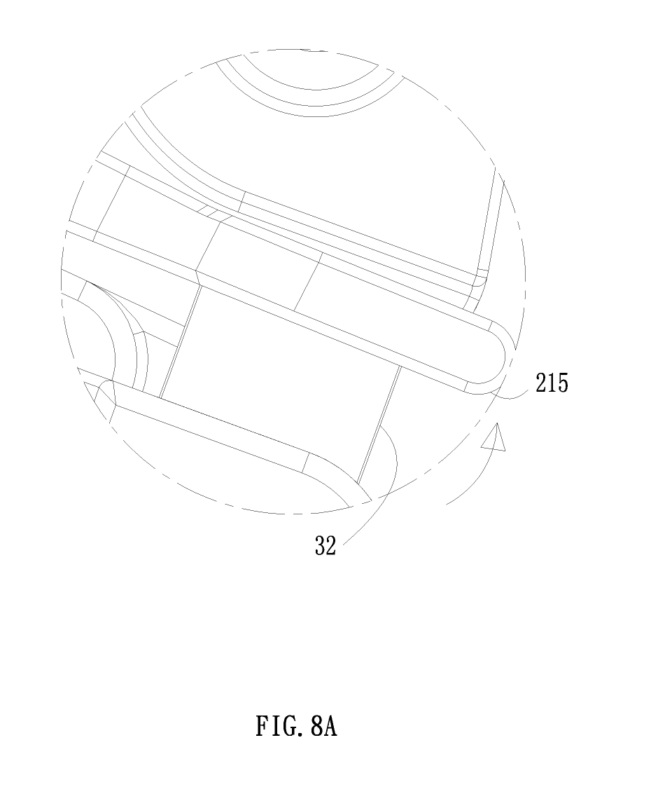

In a condition when a heel gets landed in the walking motion, as shown in FIGS. 8 and 8A, since, at this moment, a rear end of the shank frame (41) connected to the coupler (3) that is in the lower portion of the limb prosthesis is put in contact with the ground and the ground generates a reaction force in an upward direction, the upper portion of the knee joint, the knee carrier (1), the main base (2), the connection piece (11), the connection bars (217), and the axle (211) are caused to rotate clockwise about a rotation axis defined by the second threaded holes (33) and the grips (214) that are fixed to the main base (2) are also caused to do clockwise direction about the rotation axis of the second threaded holes (33) so that the push blocks (32) are pushed against by the extensions (215) of the grips (214), whereby in this moment, the one-way bearings (212) are set in a tightly gripped state to impose a constraint to the rotation direction of the axle (211), thereby achieving the purpose of preventing tumbling caused by bending of the knee joint and, at the same time when the main base (2) pushes against the push blocks (32), due to the push blocks (32) being of elasticity to absorb a terminal impact generated by the ground, comfortableness is enhanced. To this point, a complete cycle of walking motion is accomplished. (The rotation of the main base (2) is approximately 10 degrees when calculated from the initial state of being perpendicular to the ground surface; however, the present invention is not limited such a condition.)

In summary, the present invention makes use of the arrangement of one-way bearings (212) to constrain the change of direction of an axle (211) and is additionally provided with extensions (215) of grips (214) to push against push blocks (32) so as to achieve gripping and releasing by which the overall safety is enhanced, the purpose of preventing tumble is achieved, and elasticity of the push blocks (32) can be used to absorb the terminal impact force generated by the ground to improve comfortableness of walking.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A structure of tumble-resistant knee joint, comprising:
a knee carrier, which is connected to a connection piece, which comprises a shaft, and has opposite sides on which connection bars are arranged for coupling with a main base; the main base, which comprises a shaft coupling hole that is coupled to the shaft, and an axle hole, which receives an axle to extend therethrough with two bearings, two one-way bearings, two bushings, and two grips respectively fit over opposite sections of the axle extending from a center towards two opposite ends, wherein the one-way bearings constrain rotation of the axle to be in one direction and the grips each comprise an extension, the two connection bars that are coupled to the knee carrier being respectively arranged outside of the two grips, and has a lower portion in which a first threaded hole to be fixedly connected to a coupler that comprises second threaded holes; the coupler, which comprises two coupler recesses formed in a top thereof, the two coupler recesses respectively receiving therein push blocks to push against the extensions;
characterized in that an action force is generated in returning an upright condition or in a condition of a heel contacting a ground surface in a walking motion to have the main base rotated about the first threaded hole and the extensions of the grips apply a force to the push blocks of the coupler to tightly grip the one-way bearings thereby constraining rotation direction of the axle and achieving a function of preventing tumble.

2. The structure of tumble-resistant knee joint according to claim 1, wherein the extensions are rotated in a walking motion to apply a force to the push blocks of the coupler in order to tightly grip the one-way bearings thereby constraining the rotation direction of the axle to thus constrain bending of the knee joint.

3. The structure of tumble-resistant knee joint according to claim 1, wherein the coupler constrains, through the shaft coupling hole together with the coupling with the main base, the rotation angle of the axle driven by the connection bars.
